(19) United States
(12) Patent Application Publication
(10) Pub. No.: US 2026/0174988 A1
(43) Pub. Date: Jun. 25, 2026

(54) TRANSIENT AND SPECIFIC BLOOD-BRAIN BARRIER OPENING VIA BRAIN SUBSYSTEM STIMULATION

(71) Applicant: Brown University, Providence, RI (US)

(72) Inventors: Christopher Moore, Providence, RI (US); Sinda Fekir, Providence, RI (US)

(21) Appl. No.: 19/128,911

(22) PCT Filed: Nov. 13, 2023
(86) PCT No.: PCT/US2023/079522

§ 371 Date: May 9, 2025

Related U.S. Application Data
(60) Provisional application No. 63/424,668, filed on Nov. 11, 2022.

(51) Int. Cl. A61M 21/00; A61K 41/00

(52) U.S. Cl. CPC A61M 21/00; A61K 41/00; A61M 2021/0077; A61M 2210/0693

(57) ABSTRACT

Described herein are methods for stimulating specific groups of neurons in the brain, e.g. the ventral tegmental area (VTA) or its axons, wherein the stimulation can open the blood-brain barrier (BBB) with sub-millimeter and/or sub-second specificity. Stimulation of the VTA can result in opening of the BBB for drug delivery, and/or opening the BBB for clearance of “junk” and/or waste. Also described herein, are non-invasive methods to provide control to BBB permeability.

### CROSS REFERENCE TO RELATED APPLICATION

This application claims the benefit of U.S. Provisional Application No. 63/424,668 filed Nov. 11, 2022, which is incorporated herein by reference in its entirety.

### STATEMENT REGARDING FEDERALLY-SPONSORED RESEARCH

This invention was made with government support under grant numbers MH115895, NS120832 and NS125823 awarded by the National Institutes of Health. The government has certain rights in the invention.

### FIELD

This disclosure generally relates to modulating brain permeability by stimulating transient, reversible, opening and/or closing of the blood-brain barrier.

### BACKGROUND

Delivery of drugs to the central nervous system (CNS) remains a challenge in the treatment of CNS disorders such as Alzheimer's disease, Parkinson's disease, Glioblastoma and stroke. The presence of the blood-brain barrier (BBB) limits the access of drugs to the brain parenchyma. It has been reported that approximately 98% of small molecules and nearly all large therapeutic molecules, such as monoclonal antibodies, antisense oligonucleotides, or viral vectors, cannot pass through the BBB. For these reasons, delivery of drugs to the brain is still a major challenge, and recent reports indicate that less than 10% of therapeutic agents for neurological diseases enter clinical trials because of poor brain penetration. Attempts to overcome the BBB have involved increasing drug delivery of intravascularly administered drugs by manipulating the drug's permeability, and/or by local administration into brain fluids, such as the cerebrospinal fluid of brain ventricles or the interstitial fluid of brain tissue. There is a need for methods that allow for control of BBB permeability.

### SUMMARY

Described herein are methods for stimulating specific neurons in the brain, e.g. the ventral tegmental area (VTA) neurons and their axons, wherein the stimulation can open the blood-brain barrier (BBB) on sub-millimeter and/or sub-second specificity. Stimulation of the VTA can result in opening of the BBB for drug delivery, and/or opening the BBB for clearance of “junk” and/or waste.

Also disclosed herein, are non-invasive methods to provide control to BBB permeability.

Methods of modulating blood-brain barrier (BBB) permeability are described herein. In some embodiments, a method of modulating blood-brain barrier (BBB) permeability in a subject comprises stimulating a midbrain area in the subject, thereby increasing the activity of dopaminergic neurons in the midbrain of the subject; wherein said increased activity of the dopaminergic neurons in the midbrain of the subject increases blood-brain barrier permeability in the subject. Increased activity of the dopaminergic neurons in down stream projection targets of the midbrain also increase the blood-brain barrier permeability in the subject.

Also described herein, are methods of increasing penetration of a pharmacological agent across a blood-brain barrier in a subject. In some embodiments, a method of increasing penetration of a pharmacological agent across a blood-brain barrier in a subject comprises: administering the pharmacological agent to the subject; and providing at least one stimulus to a midbrain area of the subject, thereby increasing the activity of dopaminergic neurons in the midbrain area of the subject; wherein increasing the activity of dopaminergic neurons in the midbrain area of the subject increases penetration of the pharmacological agent across the blood-brain barrier in the subject. In some embodiments, the at least one stimulus can be a second pharmacological agent.

The dopaminergic neurons are in the ventral tegmental area (VTA) of the midbrain. In other embodiments, the dopaminergic neurons are in the Substantia Nigra pars compacta of the midbrain.

In some embodiments, the at least one stimulus is a behavioral stimulus. The behavioral stimulus is rewarding. In other embodiments, the behavioral stimulus leads to anticipation of a reward. In further embodiments, the behavioral stimulus is positively surprising. In some embodiments, where the behavioral stimulus is positively surprising, a Reward Prediction Error is created. In yet still further embodiments, the behavioral stimulus creates invigoration in the subject. The methods described herein can also further comprise providing a reward to the subject.

### DESCRIPTION OF DRAWINGS

FIG. 1A shows a method for measuring increased BBB permeability tracked by escape of fluorescence into the parenchyma with 2-photon imaging. FIG. 1B shows parenchymal fluorescence (bold dash-dash line) increase following calcium events in nearby VTA axons (bold solid line). FIG. 1C shows that optogenetic stimulation of these axons drives a rapid increase in axonal calcium (bold solid line) and in turn BBB permeability.

FIG. 2 shows increased BBB permeability in Primary Somatosensory Neocortex (SI) during performance of a sensory-cued task, with discrete increases associated with sensory stimulation and reward.

FIG. 3A, FIG. 3B and FIG. 3C show the close proximity of VTA axons to vessels that support its potential role in increasing BBB permeability in Forebrain targets.

FIG. 4A and FIG. 4B align data from FIG. 1 and FIG. 2, illustrating the similar timing and relative magnitude of increased BBB permeability associated with endogenous axon events (dash-dash lines), optogenetic drive (long dash-short dash-long dash lines) and reward-predictive sensory onset (solid lines).

FIG. 5 shows overlay of SI arteriole widths aligned to calcium events, optogenetic drive and sensory cue onset (measured while acquiring the BBB data in FIG. 1, FIG. 2, FIG. 4A, and FIG. 4B).

FIG. 6 shows an example of diameter changes found in SI arterioles (image on far left) by activation of Halorhodopsin-3 or Channelrhodopsin-2 expressed in smooth muscle in SMMHC-Cre mice crossed with corresponding reporter lines.

FIG. 7A, FIG. 7B, FIG. 7C, and FIG. 7D illustrate methods for obtaining 1-Photon calcium imaging of ‘spontaneous’ activity in midbrain DA axons, and using DA sensor Dlight.

FIG. 8A, FIG. 8B, and FIG. 8C show the relationship of spike-like calcium events in two axon segments proximal to and upstream of a penetrating arteriole with increased BBB permeability.

FIG. 9A shows task-related BBB increases aligned to continuous GCaMP signals. FIG. 9B shows task-related BBB increases aligned to discrete calcium spike-like events.

FIG. 10 shows galvo-mode 2P data collection when the photomultiplier is shuttered.

FIG. 11A shows MAGNIFY preparation steps. FIG. 11B shows a 3D MAGNIFY reconstruction from rVTA, with VTA axons proximal to vessels in the VTA itself.

FIGS. 12A-12C show distinct patterns in dopamine axon activity with distinct task demands, that can be employed to target BBB opening in applications. FIG. 12A shows grayscale GCaMP activity in each area across individual trials, sorted by distance (Instrumental) or time (Pavlovian) (white dot is trial onset). FIG. 12B shows the mean changes in DAT (+) axon activity in each subdivision. FIG. 12C shows the shift in reward-event peak timing under the two conditions.

FIG. 13 shows 2-photon (2P) imaging of calcium activity in distinct DS axons during an Instrumental task.

FIG. 14 shows an example of the projection distribution after transduction of axon-targeted GCaMP to DAT (+) rVTA and SNpc, showing possible targets of BBB permeability increases.

FIG. 15A and FIG. 15B show an initial test of Evans Blue Dye, comparing injections in control mice (FIG. 15A) versus concurrent injection of Lysophophatidic acid (FIG. 15B).

FIG. 16 shows enhanced signaling from a fluorescently-tagged estrogen-like compound.

FIG. 17 shows an example of identifying vessel elements.

FIG. 18 shows an example of a network output in the form of a map of vascular diameter.

FIG. 19 shows the impact of optogenetic hyperpolarization manipulation on Rule Reversal versus Rule Switching.

FIG. 20A shows several frames of fluorescence aligned to a spike event in a nearby VTA dopaminergic axon. These frames show increased BBB permeability in the form of fluorescent escape following this spike event, in an example Field of View (FOV) from 2-Photon (2P) imaging experiments in Neocortex. FIG. 20B shows BBB percent permeability change. Solid lines represent the axon in non-active frames. The dash-dash line represents spiking. The dash-dot-dash-dot line represents increased dextran signal in the parenchyma.

FIG. 21A and FIG. 21B show that VTA drive by optogenetics increases BBB permeability, hatched columns represent drive in more BBB events and faster-onset events than represented by the stippled shaded columns.

### DETAILED DESCRIPTION

Blood-brain barrier (BBB) permeability is key to delivering compounds including pharmacological agents to the brain. Described herein are methods comprising stimulating dopaminergic cells that can be used to open the BBB, providing an opportunity for pharmacological agents or the like to better enter the brain. This stimulation can be evoked behaviorally and non-invasively in human patients.

Adaptive behavior depends on body state: The correct response to a sensory input, and learning from this choice, requires a detailed understanding of current needs. Signals within vessels are a well-established source of such internal context, including hormonal, metabolic, and immunological indicators. However, the blood-brain barrier (BBB) is believed to sharply restrict such transmission, as sustained increases in permeability are a major health risk. According to the embodiments of the present disclosure, it is found that BBB is dynamic, providing increased access to body state at the time, and in Forebrain regions, where behaviorally relevant information is worth the risk. Many brain systems that index relevance may exert such control. The Ventral Tegmental Area (VTA) is an ideal candidate, as its activity signals behavioral relevance and its axons closely encircle Forebrain vessels. Two-photon imaging, optogenetics and behavioral training were used. To track rapid changes in BBB permeability, fluorescent molecules IV were injected (e.g., 70 kD Rhodamine B) and tested, and observed that they are transmitted to parenchyma of mouse Primary Somatosensory Neocortex.

During consolidation of a novel sensory association, BBB permeability increases at cue onset and reward (N=3 mice). Axons from rostral VTA neurons ramify in SI. Selective optogenetic activation in these axons drives BBB permeability, and their endogenous calcium ‘spikes’ (GCaMP) predicts it (N=53 axons, >15000 spikes). Behavioral, optogenetic and spike-aligned data show similar rapid (>250 ms) and discrete increases in permeability, suggesting a common mechanism. According to the embodiments of the present disclosure, it was found that the BBB responds rapidly, well-positioned to relay behaviorally relevant state information for integrated computations that produce adaptive behavior. These findings can have clinical import. In Alzheimer's Disease, mesocortical DA projections and the BBB are substantially altered: Failed communication between them may contribute to behavioral and health deficits. More generally, BBB compromise is central to many conditions, including addiction and sleep disorders, that are also associated with altered DA signaling.

Optimal behavior (choice, rewards) requires detailed body state information in real-time. Vascular pathways are known to send high-dimensional body state information to the Forebrain. However, the BBB is viewed as a near uniform blockade, key to protecting brain health. Described herein is a dynamic BBB granting access on millisecond time scales, opening when the risk is worth the reward. Reward events, VTA activity, and/or SNpc activity drive increases in BBB opening and/or permeability which is described herein according to the embodiments of the present disclosure. The increases in BBB permeability can be rapid.

Current and previous methods require invasive methods to provide control to BBB permeability. Described herein are non-invasive methods to provide control to BBB permeability. For example, treatment of any of a number of maladies, such as brain related cancers, can be improved by appropriately timing reward administration so that the BBB is more permeable following injection or ingestion of medication (e.g., chemotherapeutics).

### Definitions

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

“Midbrain” refers to the topmost part of the brainstem, the connection between the brain and the spinal cord. It is located below the cerebral cortex and at the topmost part of the brainstem. There are three main parts of the midbrain—the colliculi, the tegmentum, and the cerebral peduncles.

“Optogenetic” refers to activation or inhibition of the activity of specific neuron populations using light.

“Chemogenetic” refers to a technique that allows for the reversible remote control of cell populations and neural circuitry via systemic injection or microinfusion of an activating ligand.

Described herein are non-invasive methods to provide control to blood-brain barrier permeability. Providing control to blood-brain barrier permeability can include increasing permeability across the blood-brain barrier or decreasing permeability across the blood-brain barrier.

The ventral tegmental area (VTA) located in the midbrain controls diverse behaviors, including reward processing, aversion, stress modulation, drug addiction, learning, and memory. The VTA functional diversity is partly reflected by its cellular and circuit heterogeneities. The VTA is composed of ˜60% dopaminergic neurons (DA neurons), ˜35% GABAergic neurons (GABA neurons), and ˜5% glutamate neurons (glutamate neurons). It has been found that stimulating DA neurons in the VTA allows for modulation of BBB permeability.

Provided herein are methods of modulating blood-brain barrier (BBB) permeability in a subject. In some embodiments, a method of modulating BBB permeability in a subject comprises stimulating a midbrain area in the subject, thereby increasing the activity of dopaminergic neurons in the midbrain of the subject; wherein said increased activity of dopaminergic neurons in the midbrain of the subject increases blood-brain barrier permeability in the subject.

In some embodiments, a method of modulating BBB permeability in a subject comprises stimulating axons from midbrain structures, thereby increasing the activity of dopaminergic neurons in the midbrain of the subject; wherein said increased activity of dopaminergic neurons in the midbrain of the subject increases blood-brain barrier permeability in the subject.

In other embodiments, a method of modulating BBB permeability in a subject comprises stimulating axons from midbrain structures, thereby increasing the activity of dopaminergic neurons in the midbrain of the subject; wherein said increased activity of dopaminergic neurons in the midbrain of the subject and in down stream projection targets of the midbrain increase blood-brain barrier permeability in the subject.

In some embodiments of the method for modulating BBB permeability, the method allows for increasing penetration of a pharmacological agent across a BBB.

Also provided herein is a method of increasing penetration of a pharmacological agent across a blood-brain barrier in a subject, the method comprising administering the pharmacological agent to the subject; and providing at least one stimulus to a midbrain area of the subject, thereby increasing the activity of dopaminergic neurons in the midbrain area of the subject; wherein increasing the activity of dopaminergic neurons in the midbrain area of the subject increases penetration of the pharmacological agent across the blood-brain barrier in the subject.

In some embodiments of the methods described above and herein, the dopaminergic neurons are in the Ventral Tegmental Area (VTA) of the midbrain.

In some embodiments of the methods described above and herein, the dopaminergic neurons are in the Substantia Nigra pars compacta (SNpc) of the midbrain.

A subject or patient can be a mammal. A mammal can include, but is not limited to, rats, cats, dogs, deer, monkeys, apes, bats, whales, dolphins, and humans. In some embodiments, the subject is a human. In other embodiments, the patient is a human.

In other embodiments, the at least one stimulus is a behavioral stimulus. In some embodiments, the at least one stimulus is a behavioral stimulus which further comprises providing invigoration towards reward in a subject. In other embodiments, the at least one stimulus comprises providing a reward to the subject. In some embodiments, this behavioral stimulus reveals a Reward Prediction Error. In other embodiments, providing a reward to the subject provokes an emotional response that changes dopamine release in the brain, thereby allowing for modulation of BBB permeability.

In some embodiments, the at least one stimulus can be a second pharmacological agent. In other embodiments, the at least one stimulus can be administering a second pharmacological agent to the subject.

In further embodiments, the methods described herein can include providing more than one stimuli to the subject. One, 2, 3, 4, at least 1, at least 2, at least 3, at least 4, more than 1, more than 2, more than 3, or more than 4 stimuli can be provided to a midbrain of a subject according to embodiments of the present disclosure.

Described herein, behavior can include, but is not limited to, any form of motivated behavior. Motivated behavior can be behavior that is not reflexive.

Stimulating/activating the midbrain drives the output of neurons/axons. In some embodiments, behavioral tasks or experiences of specific types can also drive this pathway, i.e. it is a mode of stimulation through the perceptual system of the subject, not direct electrical or optical neural stimulation. The stimulus/stimuli can be behavioral. In other embodiments, the behavioral stimulus can be one that is rewarding, one that leads to an anticipation of a reward, one that is positively surprising (e.g. creates a Reward Prediction Error), one that leads to the subject being invigorated, i.e. wanting to do more, the like, or a combination thereof. In some embodiments, the invigoration in the subject can lead to the subject doing more to obtain another reward.

In some embodiments, the behavioral stimulus can be at least one that is rewarding, at least one that leads to an anticipation of a reward, at least one that is positively surprising (e.g. creates a Reward Prediction Error), at least one that leads to the subject being invigorated, i.e. wanting to do more, the like, or a combination thereof.

In other embodiments, the behavioral stimulus can be one or more that is rewarding, one or more that leads to an anticipation of a reward, one or more that is positively surprising (e.g. creates a Reward Prediction Error), one or more that leads to the subject being invigorated, i.e. wanting to do more, the like, or a combination thereof.

In some embodiments, the behavioral stimulus and/or the reward are provided to a subject based on the time of administration of the pharmacological agent so as to increase BBB permeability when the pharmacological agent reaches the BBB. In other embodiments, the behavioral stimulus and/or reward can be timed such that the BBB permeability is enhanced at about the time an administered pharmacological agent crosses the BBB thereby increasing penetration of the pharmacological agent across the BBB. In some embodiments, the behavioral stimulus is administered using an application while the patient is receiving the pharmacological agent (e.g., during intravenous infusion administration of a pharmacological agent). The application can be on a mobile device, an electronic device, a tablet, or the like.

In other embodiments, the at least one stimulus is deep brain stimulation.

In some embodiments, the at least one stimulus is trans-cranial magnetic stimulation (TMS).

In other embodiments, the at least one stimulus is optogenetic stimulation.

In some embodiments, the at least one stimulus is chemogenetic stimulation.

In some embodiment, the at least one stimulus is a pharmacological agent that stimulates a midbrain nucleus (e.g., VTA, SNpc) or their axons.

In yet further embodiments, the at least one stimulus can emulate the action of dopaminergic neuros or of dopamine.

In other embodiments, the pharmacological agent is a therapeutic for treatment of a central nervous system (CNS) disorder, cancer, pain, insomnia, or stroke.

In some embodiments, the CNS disorder is depression, attention deficit hyperactivity disorder (ADHD), bipolar disorder, schizophrenia, Alzheimer's Disease, Parkinson's disease, memory deficit, cognition deficit, drug addiction, migraine, seizures and/or a combination thereof.

In other embodiments, the increased BBB permeability clears debris associated with a CNS disorder or stroke out of the brain. In some embodiments, the debris can be excess, or unwanted and/or disease-causing metabolites. In other embodiments, the debris can be agglomerated protein associated with disease (e.g., tau aggregates associated with Alzheimer's Disease). In some embodiments, the debris can be excess signaling molecules such as serotonin, gamma amino butyric acid, or glutamate.

In other embodiments, the CNS disorder that can be treatable by clearing debris or junk from the brain (by modulating BBB permeability) is vascular dementia, Lewy Body dementia (or dementia with Lewy Bodies), Alzheimer's Disease, Parkinson's Disease, Frontotemporal dementia, Creutzfeldt-Jakob disease, Wernicke-Korsakoff syndrome, mixed dementia, normal pressure hydrocephalus, posterior cortical atrophy, Huntington's disease, down syndrome, and/or a combination thereof.

In some embodiments, the BBB permeability is determined by measuring a change in influx of fluorescent molecules from the vasculature into the brain. In other embodiments, BBB permeability is determined using human imaging methods, or the probability of BBB opening is inferred by measuring a change in calcium signaling. Human imaging methods can include, but are not limited to, human brain positron emission topography and magnetic resonance imaging.

In other embodiments, specific areas of the brain can be stimulated to open the blood-brain barrier with sub-millimeter and/or sub-second specificity by use of the methods described herein. The specific area of the brain can be the ventral tegmental area (VTA). Ventral tegmental area stimulation can be used to open the BBB for drug delivery, or to open the BBB for clearance of “junk” and/or waste. In some embodiments, opening the BBB for clearance of “junk” can be useful as a therapeutic approach in diseases. Diseases can include types of dementia, such as, but not limited to, Alzheimer's, vascular dementia, Lewy Body dementia (or dementia with Lewy Bodies), Parkinson's, Frontotemporal dementia, Creutzfeldt-Jakob, Wernicke-Korsakoff, mixed dementia, normal pressure hydrocephalus, posterior cortical atrophy, Huntington's disease, down syndrome, and/or a combination thereof. In other embodiments, diseases can include those with symptoms of cognitive impairment.

Methods for measuring BBB permeability include, but are not limited to, observing dye in real time coming from vessels into the brain, using new MRI sequences that measure water flux, measuring the density of staining in the brain of large macromolecular dyes injected IV, or a combination thereof. Measuring the density of straining in the brain of large macromolecular dyes injected IV can be a post mortem, non-dynamic measurement.

The methods described herein, can measure BBB permeability by observing dye in real time coming from vessels into the brain, measuring the density of staining in the brain of large macromolecular dyes injected IV, or a combination thereof.

In some embodiments, the methods described herein can determine BBB permeability, by measuring a change in influx of fluorescent molecules from vasculature into the brain. In other embodiments, the BBB permeability can be determined by measuring a change in calcium signaling.

Calcium permeability is often a marker of increased BBB permeability, and is one indicator opening/increased BBB permeability has occurred.

Reward behavior can modulate specific blood-brain barrier opening(s). In some embodiments, a reward is given with or without direct brain stimulation, and the result is a specific BBB opening. In other embodiments, a long-term application can comprise giving a patient/person undergoing chemotherapy for a brain tumor an application that simulates a type of emotional response, and that response can help target the administered chemotherapy across the BBB and into the brain.

Described herein are methods for opening a blood-brain barrier comprising: providing at least one stimulus to a specific area of a brain; and stimulating the specific area of the brain thereby opening the blood-brain barrier. In some embodiments, the specific area of the brain is the ventral tegmental area. Stimulating the specific area of the brain can increase ventral tegmental area dopamine neurons

In other embodiments, the specific area of the brain is the Substantia Nigra pars compacta. Stimulating the specific area of the brain can increase activity in Substantia Nigra pars compacta dopamine neurons. The at least one stimulus can be a behaviorally relevant stimulus (or a behavioral stimulus). In other, embodiments the stimulus can be external or internal.

Also described herein are methods of treating a disease comprising: providing at least one stimulus to a ventral tegmental area of a brain; and stimulating the ventral tegmental area of the brain to open the blood-brain barrier for the clearance of “junk” associated with the disease. In some embodiments, the disease is Alzheimer's.

Described herein are also methods of treating a disease comprising: providing at least one stimulus to a Substantia Nigra pars compacta area of a brain; and stimulating the Substantia Nigra pars compacta area of the brain to open the blood-brain barrier for the clearance of “junk” associated with the disease. In some embodiments, the disease is Alzheimer's.

Further described herein are methods of opening the blood-brain barrier, comprising: providing at least one stimulus to a specific area of a brain; stimulating the specific area of the brain thereby opening the blood-brain barrier; and providing a reward to elicit an emotional response, wherein the emotional response targets a pre-administered therapeutic across the opened blood-brain barrier and into the brain. In some embodiments, the pre-administered therapeutic is chemotherapy.

Also described herein are methods for increasing permeability of a blood-brain barrier, the method comprising: providing at least one stimulus to a specific area of a brain; and stimulating the specific area of the brain thereby increasing permeability of the blood-brain barrier. In some embodiments, the specific area of the brain is the ventral tegmental area. The at least one stimulus can be a behaviorally relevant stimulus (or a behavioral stimulus). In other, embodiments the stimulus can be external or internal. Stimulating the specific area of the brain can increase activity in ventral tegmental area dopamine neurons.

Also described herein are methods for increasing permeability of a blood-brain barrier, the method comprising: providing at least one stimulus to a specific area of a brain; and stimulating the specific area of the brain thereby increasing permeability of the blood-brain barrier. In some embodiments, the specific area of the brain is the Substantia Nigra pars compacta. The at least one stimulus can be a behaviorally relevant stimulus (or a behavioral stimulus). In other, embodiments the stimulus can be external or internal. Stimulating the specific area of the brain can increase activity in Substantia Nigra pars compacta area dopamine neurons.

### EXAMPLES

### Example 1

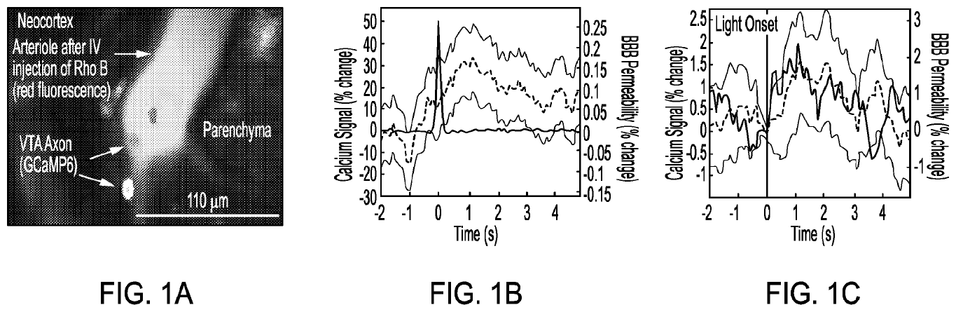

To measure VTA and BBB activity, a large fluorescent molecule (e.g., 70 kD Rhodamine B) is intravenously (IV) injected in an awake mouse whose VTA axons express a calcium indicator (GCaMP) and a cation-passing opsin (Chrimson). Using 2-Photon (2P) imaging in Primary Somatosensory Neocortex (SI), increased BBB permeability is tracked by escape of fluorescence into the parenchyma illustrated in FIG. 1A. FIG. 1B shows that parenchymal fluorescence (bold dash-dash line) increases following calcium events in nearby VTA axons (bold solid line; mean signal changes triggered on 2550 calcium events in 32 endings across 3 Fields of View (FOV) in 3 mice: 95% Confidence Intervals are shown). FIG. 1C shows, for the same FOVs, that optogenetic stimulation drives a rapid increase in axonal calcium (bold solid line) and in turn BBB permeability (mean 67 trials/mouse, because data are highly similar for 2 seconds(“s”) and 20 s red light exposures, these trials are averaged; light onset at 0). These complementary approaches demonstrate that VTA inputs can drive rapid increases in BBB permeability.

### Example 2

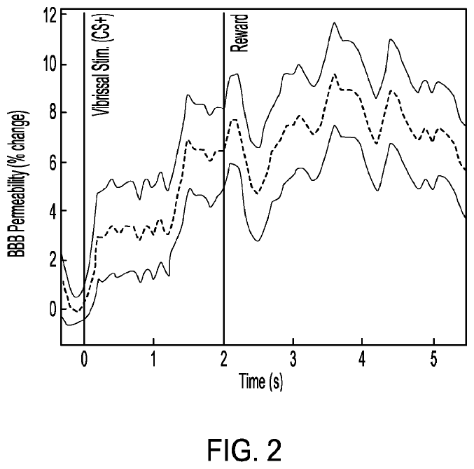

In a sensory cued task, water-deprived mice lick in response to detection of vibrissal motion (3 mice/FOVs, 120-140 trials/mouse). FIG. 2 shows increased BBB permeability in Primary Somatosensory Neocortex (SI) during performance of the sensory-cued task, demonstrating that BBB permeability can occur on a behaviorally-relevant, sub-second time scale.

### Example 3

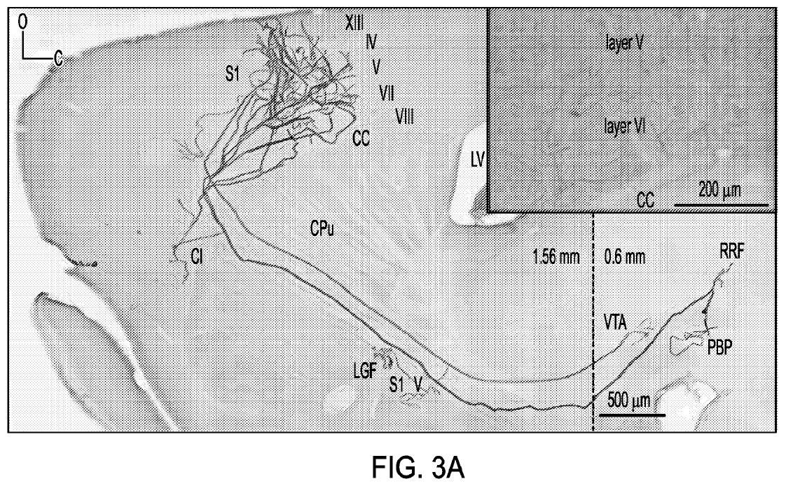

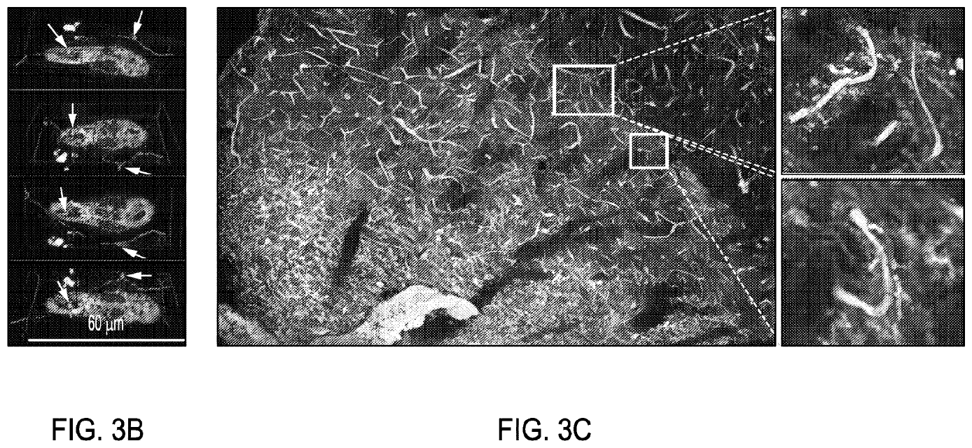

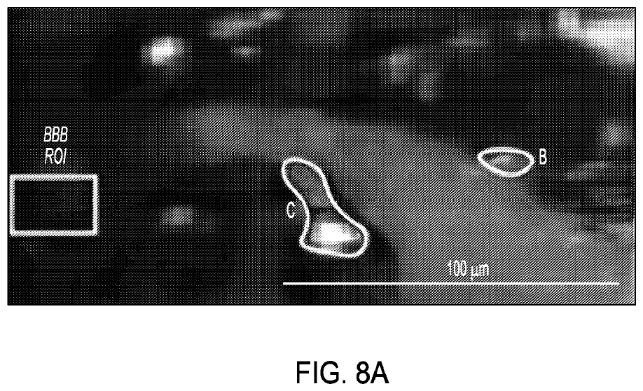

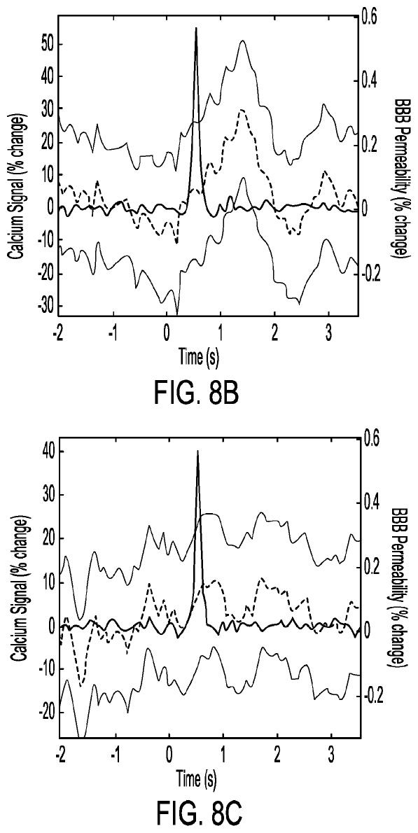

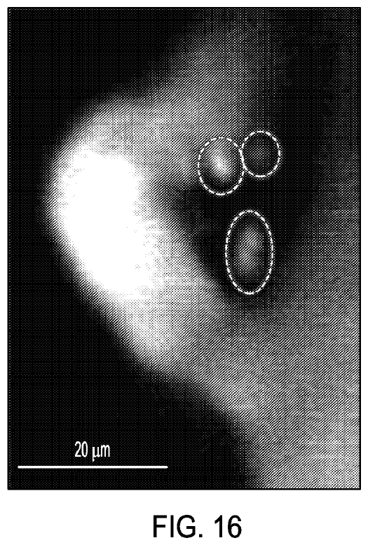

FIG. 3A shows single cell fills in Rostral VTA and ParaBrachial Pigmented Nucleus that send specific axonal projections that arborize in mouse SI. At this anterior position (referred to as “rVTA”), ˜10% of cells project without significant collateralization to SI. This mouse finding is consistent with VTA projections in other species and data. In Dopamine Transporter (DAT)-Cre mice floxed constructs were transduced in rVTA by AAV delivery and imaged in vivo using 2P. In all mice (N=21), axon projections are found to SI that are proximal to penetrating arterioles at the Layer I-II border (e.g., see FIG. 1, FIG. 8 and FIG. 16). FIG. 3B shows an example of axon-capillary proximity from superficial SI, the rVTA axon is (labeled with tdTomato) and the capillary wall (lectin antibody; DAPI). Arrows indicate varicosities, the open arrowhead a close vessel contact. FIG. 3C shows DAT (+) axons labeled with GCaMP6 after a midbrain transduction of DAT (+) cells (eGFP antibody). Boxed insets show axons in DS proximal to and encircling thin caliber vessels.

### Example 4

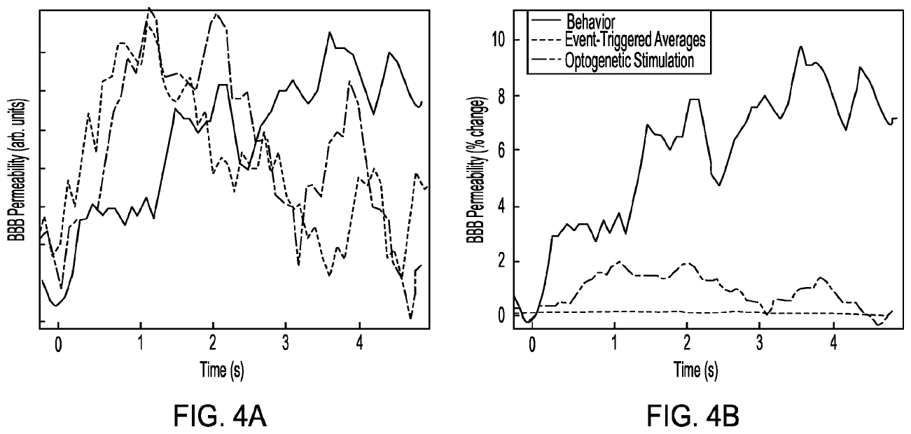

FIG. 4A and FIG. 4B aligns data from FIG. 1 and FIG. 2 showing increased BBB permeability associated with endogenous axon events (dash-dash lines), optogenetic drive (long dash-short dash-long dash lines) and reward-predictive sensory onset (solid lines). While each condition shows rapid BBB dynamics, and the endogenous axon calcium events and optogenetic-driven responses have a highly similar time course (FIG. 4A), optogenetic light that synchronously activates a field of VTA endings drives a several-fold greater increase in BBB permeability. Behavior-associated changes in the BBB are, in turn, an order of magnitude larger than those observed with VTA optogenetic drive (FIG. 4B), possibly reflecting additional kinds of drivers and/or greater VTA recruitment.

### Example 5

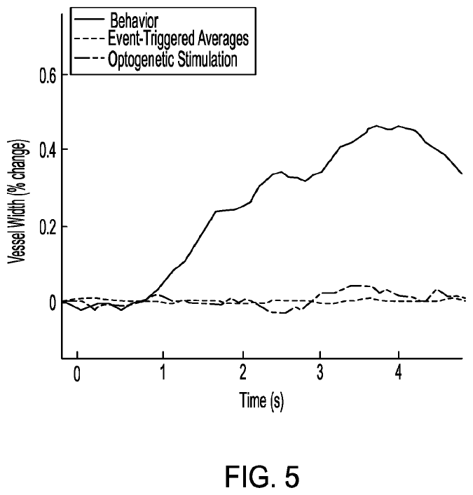

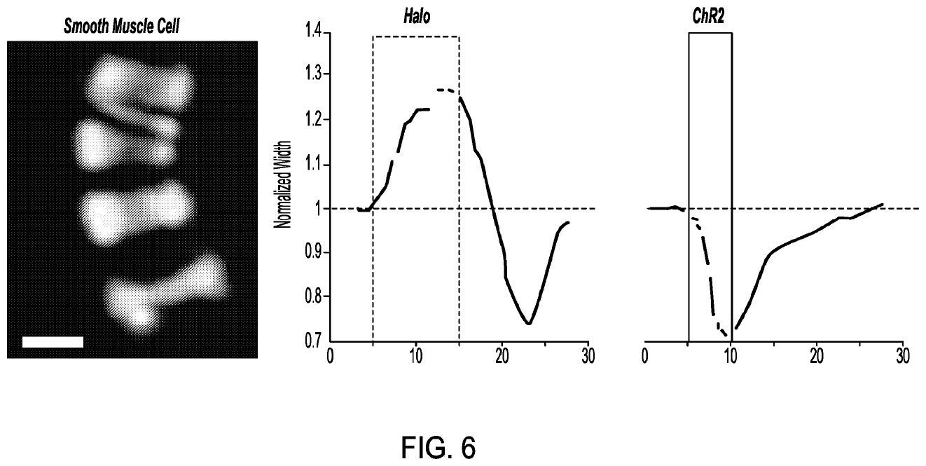

FIG. 5 shows overlay of SI arteriole widths aligned to calcium events, optogenetic drive and sensory cue onset (measured while acquiring the BBB data in FIG. 1, FIG. 2, FIG. 4A, and FIG. 4B). Single events and optogenetic activation of a field of VTA axon segments do not drive appreciable dilations. Further, even with behaviorally-relevant sensory drive, arteriole expansion initiates ˜1s post-stimulus onset, after increased BBB permeability is observed (FIGS. 4A, 4B: onset <500 msec post-stimulus). However, as suggested by the distinct BBB increase during behavior at ˜1s, a VTA-dilation-BBB relationship can exist. FIG. 6 shows an example of diameter changes found in SI arterioles (image on far left) by activation of Halorhodopsin-3 or Channelrhodopsin-2 expressed in smooth muscle in SMMHC-Cre mice crossed with corresponding reporter lines.

### Example 6

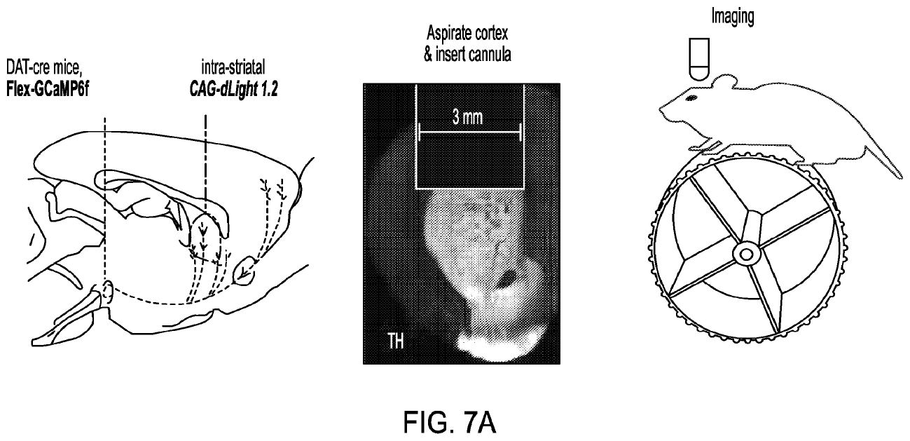

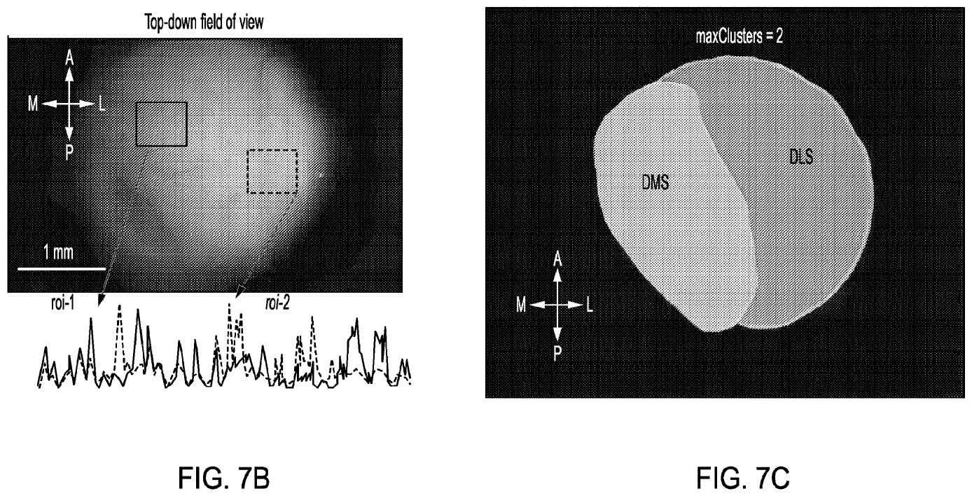

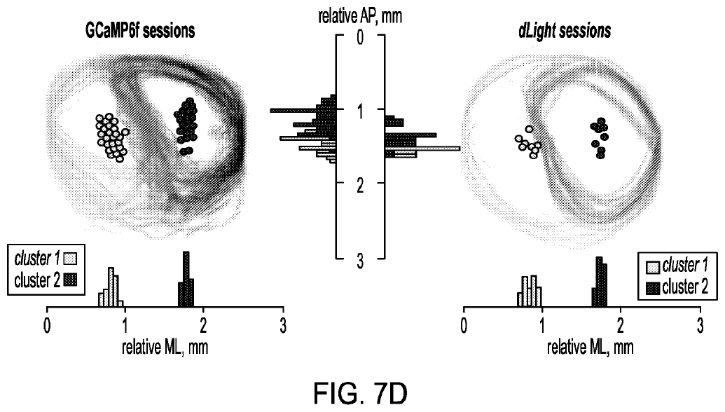

Several lines of evidence identify DorsoMedial and DorsoLateral Striatum (DMS and DLS) as functionally-distinct sub-regions. FIG. 7 illustrates these subdivisions in data obtained using 1-Photon calcium imaging of ‘spontaneous’ activity in midbrain DA axons, and using DA sensor Dlight.

### Example 7

In support of selectivity (Tuning), FIG. 8A shows the relationship of calcium events in two axon segments proximal to and upstream of a penetrating arteriole. Both axons showed similar events and firing rates, but events in axon ‘B’ (FIG. 8B) predict a discrete increase in BBB permeability (N=323 events), while those in axon ‘C’ (FIG. 8C) do not (N=292 events). It is observed that increased BBB permeability can precede, and therefore predict local calcium events, which can reflect influence of vascular signals on local VTA endings.

### Example 8

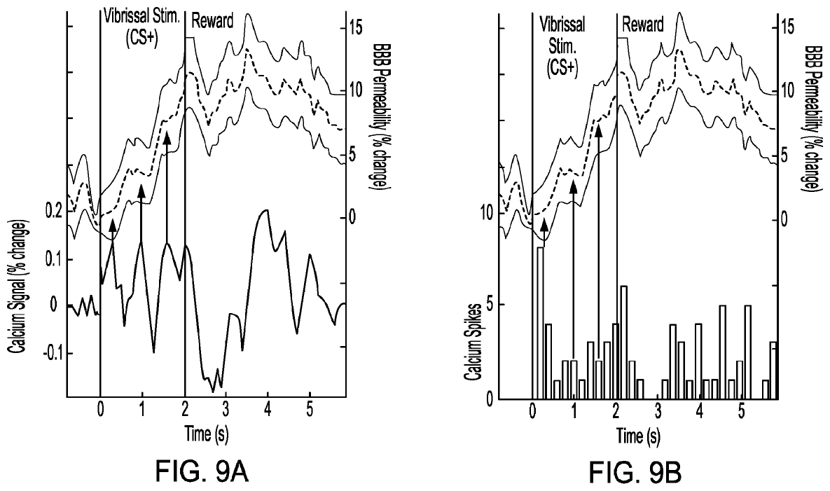

Calcium events in rVTA inputs predict increased BBB permeability with 2P imaging of axon-targeted GCaMP. FIG. 9A shows task-related BBB increases aligned to continuous GCaMP signals, and FIG. 9B shows task-related BB increases aligned to discrete calcium spike-like events (N=1 mouse/FOV with 6 axon segments). Both metrics show rVTA axon activity immediately preceding the initial increase in BBB permeability, but display different patterns of activity, each potentially predictive of different BBB components.

### Example 9

rVTA axons are optogenetically driven while measuring their calcium activity and BBB permeability with 2-Photon Optogenetic Stimulation (2P). In all optogenetic experiments, the relationships between endogenous axon segment activity and BBB dynamics are measured, using a separate imaging session to obtain these data if necessary. Optogenetic stimuli at psuedorandomly chosen are presented, counterbalanced inter-stimulus intervals from 7 to 20 seconds. Optogenetic drive lasts 2 and 4 seconds, and be presented at 7 equally-spaced intensities between a minimum of 1 mW and a maximum that is the lowest photon density to drive rapid onset calcium events in the maximal number of axons within a FOV.

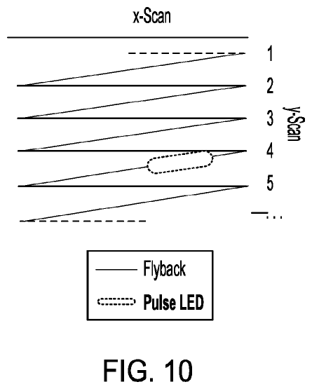

Multiple steps are taken that minimize optogenetic-light contamination of 2P GCaMP imaging. First, Chrimson is used, whose red-shifted sensitivity allows drive with 625 nm stimulation, which can be filtered for contamination of the GCAMP signal. Further, optogenetic light is presented during ‘flyback’ of galvo-mode 2P data collection when the photomultiplier is shuttered as diagrammed in FIG. 10.

### Example 10

Systematic Determination of Axon-Vessel Appositions: First, the prediction that VTA projections to SI are composed of DAT (+), VGLUT2 (+), and VGLUT2/DAT (+) cells, as in mPFC can be tested. To test this prediction, loxP-Cre or Flpo-dependent viral expression in DAT-Cre, VGLUT2-Cre and VGAT-Flpo mice can be used. If glutamatergic or GABAergic SI input is observed, INTERSECT2.0 viral approaches can be used to determine co-expression.

Second, the prediction that all VTA subtypes that project to an area project to penetrating arterioles, capillaries and post-capillary venules (<1 μM apposition), and, that single axon trajectories interact with multiple vascular elements can be tested. That varicosities appose each vessel element type can be tested. To analyze these data, resampling is performed by separating axon and vessel color channels, and randomly overlaying them to re-calculate null proximity with structure, and determining the pixel-level proximity probability with bootstrapped resampling.

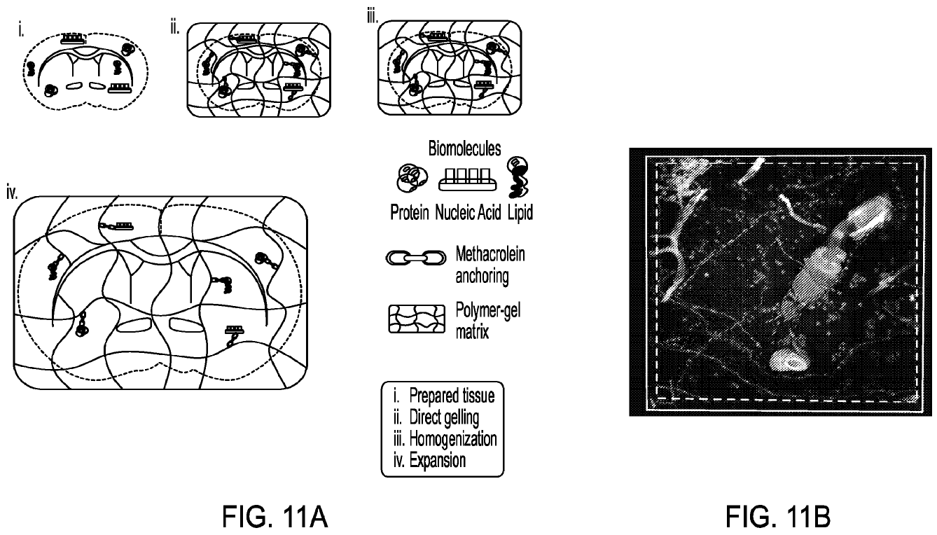

A key method showing close apposition of VTA axon segments to vessels is MAGNIFY. This strategy does not require a special fixation or attachment step to anchor biomolecules, and preserves proportional relationships in fine cellular structure up to 11-fold expansion. As such, MAGNIFY allows enhanced judgement of proximity using confocal imaging. FIG. 11A shows its preparation steps, FIG. 11B shows a 3D MAGNIFY reconstruction from rVTA, with eGFP antibody, DAPI, SMA (muscle/arteriole) and lectin (vessel). In this projection, DAT (+) axon trajectories, varicosities, and their proximity to distinct vascular elements are seen. Post mortem brains are tested with antibodies for varicosity labeling (e.g., RIM64), and as needed for eGFP (FIG. 11B) tdTom (FIG. 3), DAT, TH, Lectin, and SM. Pre-expansion confocal measurements are compared with MAGNIFY data to calculate fold expansion and track for any distortion: It is confirmed that tdTom-labeled VTA projections are not distorted by expansion. Third, conventional confocal imaging is used for comparison with these other analyses.

### Example 11

Testing the VTA-BBB Hypothesis in Dorsolateral (DLS) versus Dorsomedial Striatum (DMS). The studies can be conducted on Dorsal Striatum (DS). First, DS is central to optimal behavior. Second, DS subdivisions receive robust and distinct projections from SI and dmPFC, allowing to test if there are similar VTA-BBB impacts on corticostriatal partners that work together. Third, DS allows for systematically testing whether all midbrain dopaminergic axons impact BBB permeability in a common target equally, or whether those originating in the SNpc are distinct. Fourth, DAT (+) axon activity differs in Dorsolateral (DLS) versus Dorsomedial Striatum (DMS) as a function of task requirement: This spatiotemporal variation in DAT (+) axon activity provides a robust backdrop for testing its association with permeability.

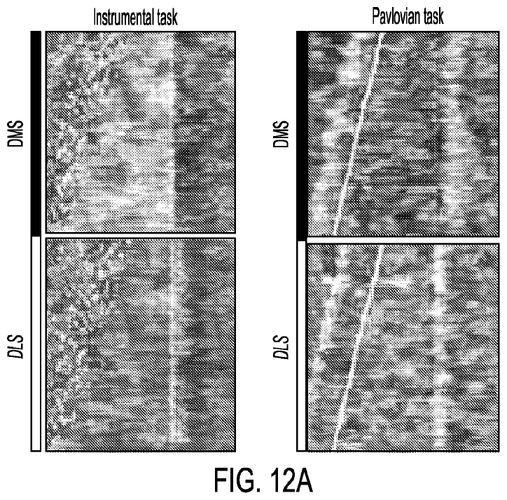

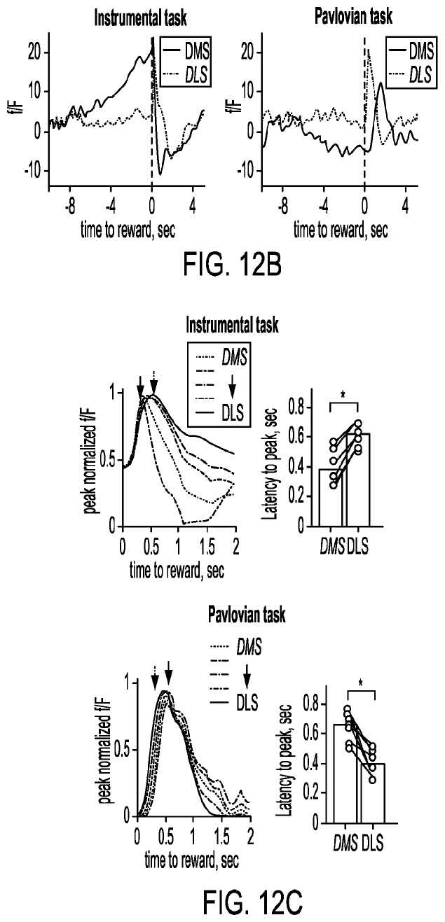

A basic behavioral design can be employed, in which sensory input (e.g., an auditory tone) cues trial onset, and increasing frequency tone steps indicate proximity to reward. An “Instrumental” variant of this task requires locomotion, and tone increments indicate progress to the reward. In this task, as shown in FIG. 12, axon firing in DMS ramps up during approach to reward, at which point an additional discrete activity burst occurs followed by a later, weaker peak in DLS. The “Pavlovian” variant does not require locomotion, and sensory increments indicate the time to reward. Under these conditions, DMS DAT (+) axon activity steadily decreases, and the reward-related burst initiates in DLS and then propagates to DMS. FIG. 12A shows grayscale GCaMP activity in each area across individual trials, sorted by distance (Instrumental) or time (Pavlovian) (white dot is trial onset). FIG. 12B shows the mean changes in DAT (+) axon activity in each subdivision. FIG. 12C shows the shift in reward-event peak timing under the two conditions.

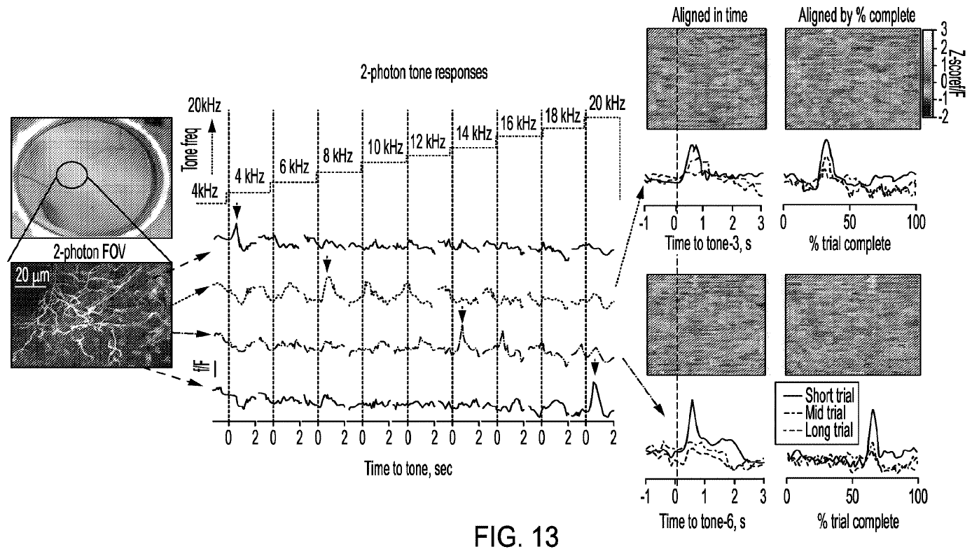

FIG. 13 shows 2P imaging of calcium activity in distinct DS axons during an Instrumental task. Within the macrostructure of the DAT (+) axon population shown in FIG. 12, refined tuning of individual axons is found, for example, to specific tone transitions. This precision in tuning deomonstrates that distinct axon segment calcium events drive increased BBB permeability.

Correlative imaging and direct optogenetic stimulation testing of BBB permeability using the imaging approach is conducted, with distinct viral transductions of axonal GCaMP and opsins into SNpc or VTA. In distinct experiments, virus is transduced in dorsal or ventral SNpc, to target both matrix and striosomes of DS, or in VTA. These targets are studied in isolation in a subset of mice and in combination, with axon targeted GCaMP and the red indicator JRGECO in SNpc or VTA simultaneously. The dual color strategy allows direct comparisons of DAT (+) axon activity in the same FOV.

### Example 12

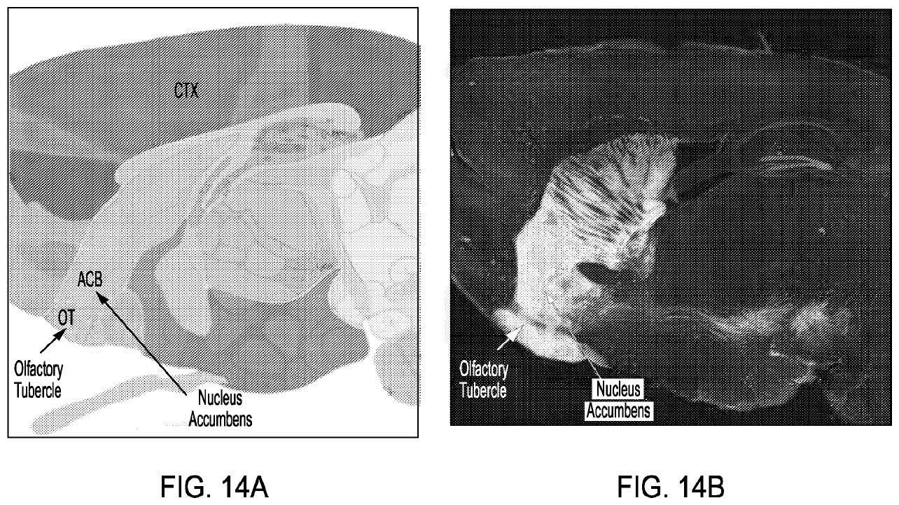

Testing the VTA-BBB Hypothesis to test whether rVTA activity drives increased BBB permeability in downstream targets, rVTA activity is driven and classical methods are conducted for monitoring whole-brain BBB permeability. FIG. 14 shows an example of the projection distribution achieved after transduction of axon-targeted GCaMP to DAT (+) rVTA and, in this case, modest involvement of SNpc. This image illustrates areas that can show robust increases in BBB permeability: Nucleus Accumbens (ACB) and Olfactory Tubercle (OT), in addition to DS, dmPFC and S1.

Multiple stimulation approaches can be used to test whether stimulation in rVTA drives increased BBB permeability in all targets. First, rVTA transduced with Chrimson can be directly optogenetically stimulated, in VGLUT2-Cre, DAT-Cre and, potentially, VGAT-Flpo mice. Second, these same afferents can be chemogenetically driven using BL-OG construct LMO7. If any significant difference is observed in BBB permeability between opto-and chemogenetic drive, using this BL-OG molecule also applies optogenetic drive in rVTA and in target structures, to test for uniformity of method. For comparison with the substantial literature examining VTA electrical stimulation and behavior, afferents can also be driven using direct electrical bipolar stimulation. Experiments can be performed on awake, quiescent mice in which arousal indices can be tracked: As necessary, parallel studies can be conducted in lightly isofluorane anesthetized mice (˜0.5%), that do not show behavioral fluctuations with stimulation, to diminish the impact these can have. Parallel fiber optic and chemogenetic stimulation can be performed in null mice transduced with inactive opto-and chemogenetic variants.

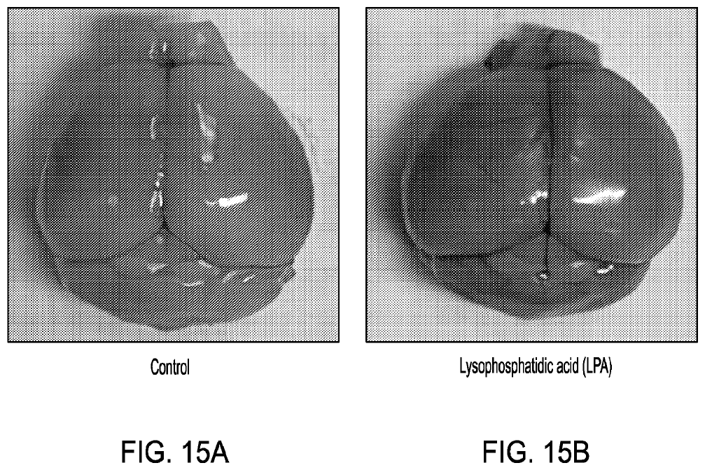

Evans Blue dye (EBd) transmission to the brain test is used, and test fluorescent marker Rhodamine 123 transport. For both approaches, fluorescence ratio of vasculature to brain is calculated, in actively driven versus control mice and regions that do not receive VTA input in a given mouse. Lectin fluorescence (e.g., FIG. 3 and FIG. 11) is used to identify and exclude vessels and to normalize for BBB surface area density. Using standard confocal imaging of sagittal sections, in each mouse, is conducted a 1st order wide-field determination of the termination patterns of that transduction, the specific position of somata transduced, and reconstruction of the fiber optic/electrode position, aided by leaving these probes in the brain during perfusion, and as needed by making small electrolytic lesions. FIG. 15A and FIG. 15B show an initial test of EBd, comparing injections in control mice (FIG. 15A) versus concurrent injection of Lysophophatidic acid (FIG. 15B), which causes generalized BBB permeability.

### Example 13

### Systematic Testing of the VTA-BBB Permeability

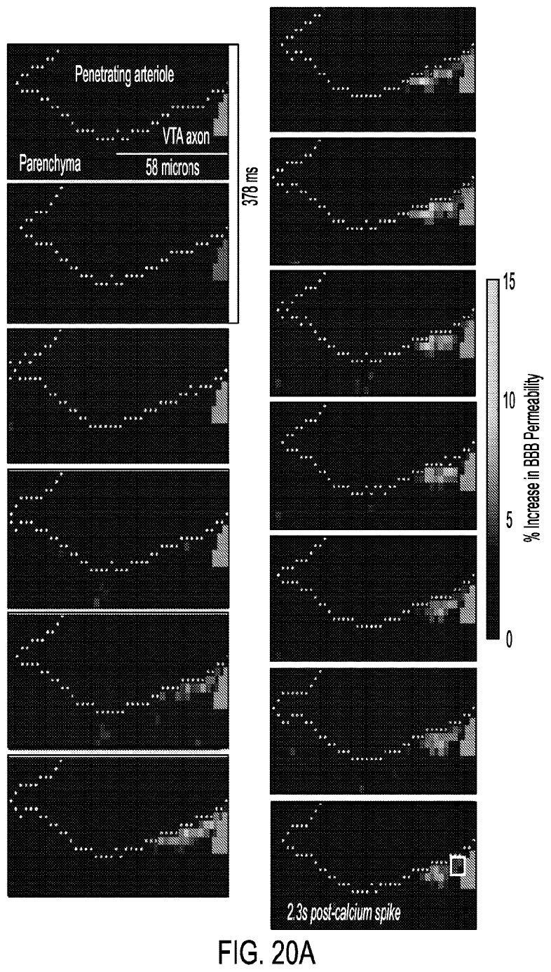

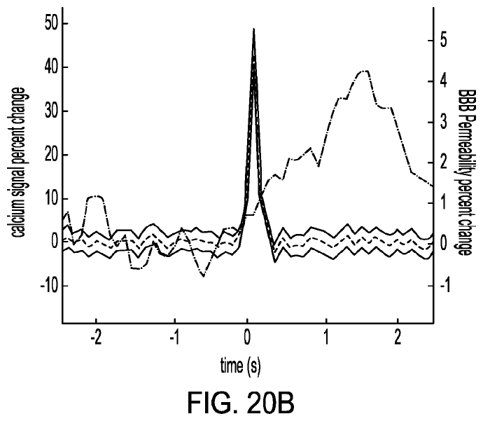

This method allows light microscopy to be used to test VTA axon proximity to arterioles, capillaries and venules. BBB permeability increases immediately proximal to VTA axons after they spike. BBB permeability increases in/near the Virchow-Robin space following calcium spike events in VTA axons along the length of the penetrating arteriole (FIG. 1, FIG. 4A, FIG. 4B, FIG. 8, FIG. 9A, and FIG. 9B). In this experiment is it found that BBB permeability also increases immediately proximal to active VTA axons. FIG. 20A shows several frames in an example Field of View (FOV) from the 2-Photon (2P) imaging experiments in Neocortex. In these studies, the calcium indicator GCaMP6s in VTA axons is expressed and a 70 kD dextran IV is injected. This large molecule does not significantly enter parenchyma without increased permeability, thereby providing a fluorescent index of VTA impact on the BBB.

In the FOV, the white dots mark the edge of a penetrating arteriole, its internal fluorescence masked for ease of visualization. Image frames show the FOV before and immediately after calcium spike events in the VTA axon (N=mean of 31 aligned calcium events from the axon; solid lines represent the axon in non-active frames, the dash-dash line represents spiking). The dash-dot-dash-dot line represents an increased dextran signal in the parenchyma, the time series shows these changes in the gold region of interest in the last panel, arteriole and axon signals were excluded from analysis.

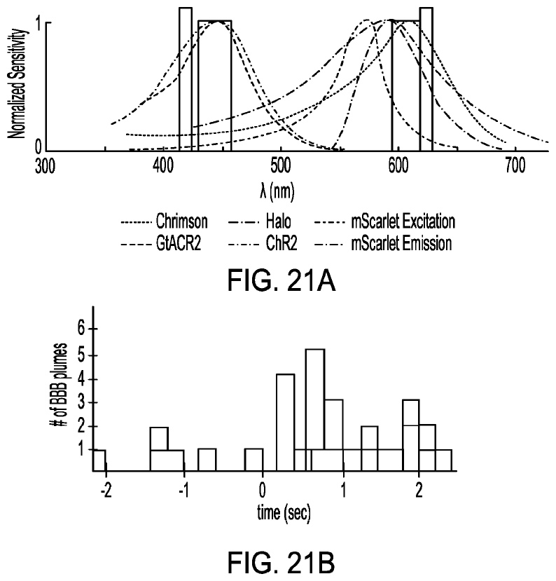

As shown across images and in the time series, BBB permeability increases adjacent to the active axon. This rapid increase in BBB permeability then spreads laterally across the domain local to the driving axon within a few hundred milliseconds. These data, together with the optogenetic and behavioral data, suggest that VTA events and/or increases in DA neuronal activity in the midbrain drive BBB increases both distally and at the point of axon-vessel contact. Thus, VTA axon signaling to vessels/the BBB can be targeted to creating focal and broader impact (e.g., in a micro-domain and across a Neocortical column). FIG. 21A and FIG. 21B show that VTA drive by optogenetics increases BBB permeability, hatched columns represent drive in more BBB events and faster-onset events than represented by the stippled shaded columns.

### General Methods

### 2P Imaging

Genetically-encoded calcium indicators are targeted using the GAP43 trafficking motif that drives expression preferentially in axons, as shown in FIG. 1, FIG. 8, FIG. 11 and FIG. 16. Volumes are sampled using a liquid Optotune lens, enhancing coverage and, after motion correction, precision in sampling the same axonal sub-segments and ROIs. To date, axon-targeted GCaMP6s have been employed: At the outset, a systematic comparison of indicator sensitivity/speed in target areas is conducted to ensure the highest SNR for each.

Monitoring BBB permeability on 2 time courses with 2P imaging First, parenchymal fluorescence of IV injected molecules at <100 msec resolution is tracked. Signal intensity throughout each FOV is measured in 10 μm² ROIs, using in-house methods for z-dimension motion correction to assure consistent position alignment and vascular wall removal, mitigating contamination from dilation/constriction (Fekir, Klein and Moore, unpublished). Second, fluorescent conjugate density is measured in endothelial cell walls and in parenchyma, across ˜tens of minutes, in high-resolution anatomical stacks centered on the dynamically-imaged FOV, taken before and after each dynamics scan session.

Four fluorescent molecules whose transmission across the BBB (when it occurs) is mediated by distinct mechanisms are tested.

Rhodamine B Dextran (70 kD), shown throughout, does not cross the BBB unless paracellular permeability is increased. Access of this molecule, and its speed in experiments, suggest a paracellular VTA-BBB mechanism that is more rapid than multi-stage transcytosis or transmembrane diffusion. The red shifted emission of Rhodamine B facilitates separation from simultaneous GCaMP imaging using common 2P excitation at 960 nm.

Rhodamine 123 is used as a functional assay for proteo-glycoprotein efficacy in maintaining BBB patency including by tracking its accumulation in parenchyma ex vivo following in vivo intravenous (IV) injection.

Fluorescence-Tagged Albumin is used as a functional assay for receptor-mediated transcytosis and/or adsorptive transport across the BBB, and provides robust individual molecular signals using 2P imaging in vivo. BSA-Alexa488 and BSA-Alexa594 is employed, for optimal comparison to prior studies, and BSA-Rhodamine B (Nancos) for parallelism to our other testing.

Fluorescence-Tagged Estrogen (17-Estradiol Jena Bioscience) Estrogen crosses the BBB through simple diffusion and, likely, receptor-mediated mechanisms. It also acts on endothelium, increasing BBB patency and recovery from insult in part by restoration of tight junctions that mediate paracellular transport. These features make it a conservative and ethologically-relevant probe of the VTA-BBB Hypothesis. FIG. 16 shows an initial viability test of this construct for 2P imaging (900 nm) with tail vein injection, circled portions represent axon segments.

### Sex as a Variable

Hormone delivery to the brain can be impacted by BBB permeability. Equal numbers of male and female mice are used in all experiments and, in females, estrous is tracked.

### Identifying Selected Vessel Elements

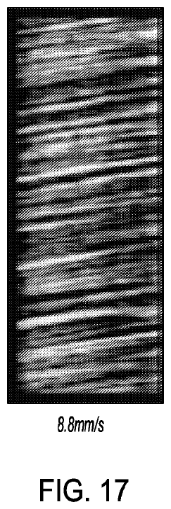

While often treated as a uniform barrier, distinct BBB dynamics have been observed in specific vessel elements: Permeability through pial and penetrating arterioles is selectively impacted by alterations in the apolipoprotein M transporter, while Transferrin-dependent transport of nanoparticles occurs at post-capillary venules. In addition to a computational approach, vessels by cardinal anatomical features are identified, injection of Alexa Fluor Hydrazide 633, which labels arteries selectively, and track the velocity and direction of red-blood cell shadows (>1 kHz line scan). FIG. 17 shows an example of such obtained measurement. Experiments are also conducted using non-floxed VTA transduction in transgenic lines crossed with fluorescent reporters, to selectively label arterial endothelium (endothelial label: bmx(PAC)-Cre) and smooth-muscle (predominantly localized to arteries, SMMHC-Cre, FIG. 6). Venules can be identified by size, shape and flow, but if needed selective venous endothelial tagging (Gm5127(BAC)-CreERT2) can be explored.

### Network Graph/Machine-Learning Pipeline for Vascular Analysis

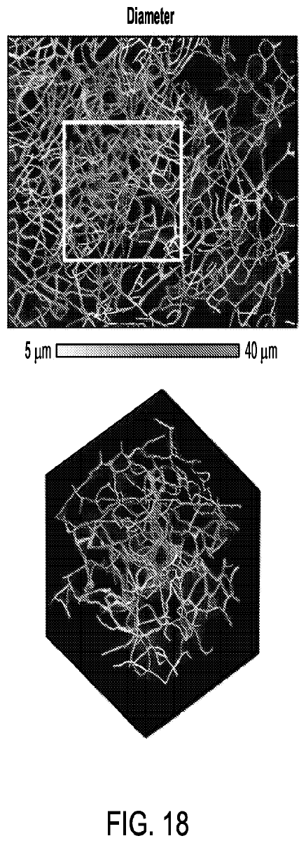

A computational approach for vascular element identification developed by Dr. Jonghwan Lee is adapted and used. This approach automates identification of vessel elements (arterioles, capillaries and venules), including the branch-order depth of each capillary segment. Given that ˜85% of brain vascular length resides in capillaries, their substructure can be important to BBB dynamics. This approach applies 3 stages of tailored Convolutional Neural Networks for enhancement, segmentation and gap-correction of the vessel angiogram. A network vascular graph is then constructed. FIG. 18 shows one example of the network's output, a map of vascular diameter.

### Sub-Region and Cell-Type Targeting in VTA

DAT (+) projections are obtained following viral injections in the focus referred to as ‘rVTA,’ which overlaps the Rostral VTA and ParaBrachial Pigmented Nucleus. One to four injections of AAV (0.2 μl) is delivered centered on AP −2.8, ML 0.5 and DV −4.4. To ensure mPFC projection labeling, more medial (M/L: 0.34 to 0.5) is injected and to target DS, more lateral (M/L-0.8) is also injected. Post hoc, brain slices are harvested and somatic transduction to VTA nuclei is localized as in. To test/confirm glutamatergic and GABAergic projection cells reaching SI and dmPFC, viral transgenic targeting in the VGLUT2-Cre and VGAT-Flpo mice is used, including if warranted the INTERSECT2.0 approach to test co-expression, to build on and compare with prior behavioral/optogenetic studies.

### Quiescence and Sensory-Cued Behaviors: Context for Testing the VTA-BBB Hypothesis

‘Quiescent’ Mice in FIG. 1 and FIG. 8, show that VTA activity can increase BBB permeability are acquired in mice well-adapted to head-posting on a locked running wheel. Such quiescence is a key ethological condition: Processes such as memory formation of recently completed behaviors can occur during a pause in learning, and be facilitated by well-time state information from the vasculature. Further, using quiescence allows decoupling of BBB observations and behavior-related events such as vasodilation (FIG. 5).

### Sensory-Cued Tasks: ‘Pavlovian’ and Instrumental

During tasks where sensory cues predict upcoming rewarding or aversive stimuli, in the ‘Instrumental’ version of these tasks, timed motor action is required to elicit or avoid outcomes: In the ‘Pavlovian’ version, the cue simply indicates an obligate subsequent event, independent of action (FIG. 12). To help ensure bidirectional VTA engagement, cue validity is a high-probability regime and suprathreshold sensory intensity is used.

Activity in DS, and in SI neurons that project to it, predicts task success in mice trained to lick in response to suprathreshold vibrissal stimuli. This task was employed extensively (e.g. see FIG. 2, FIG. 9A, and FIG. 9B). Head-posted mice are trained to respond to strong vibrissal stimuli and not invalid auditory distractors (10 kHz) or vice versa. Cohorts are trained with a Pavlovian design where water rewards are presented at fixed latencies to sensory onset, or an Instrumental design, where reward requires locomotion after sensory onset, with increasing sinusoidal vibrissa deflections indicating progress to reward (FIG. 13). Vibrissal stimuli step from 40 Hz to 320 Hz, an ethologically valid regime. To engage VTA glutamatergic projections that can respond to aversive stimuli and/or valid cues, a cohort is trained that vibrissal stimuli predict air puff (paralleling ‘Pavlovian’reward).

Rule Switching between Vibrissal and Auditory Cue Validity

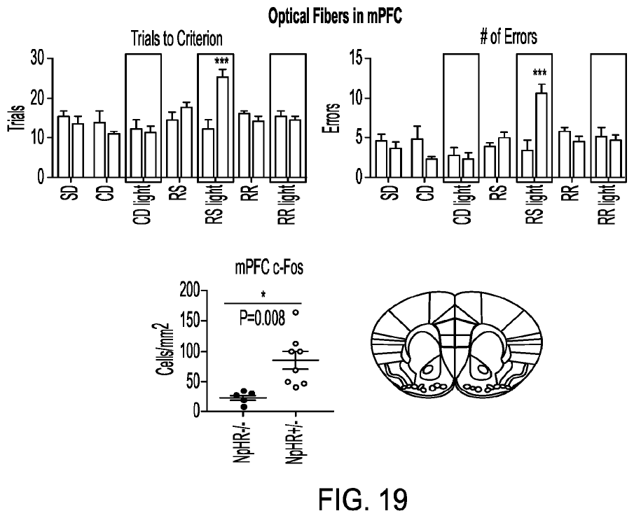

The mPFC is key to successfully shifting to a previously invalid sensory cue. Optogenetic hyperpolarization of interneurons in mPFC delays transition from a previously valid reward cue to newly valid sensory modality: FIG. 19 shows the impact of optogenetic hyperpolarization manipulation on Rule Reversal versus Rule Switching.

This disclosure includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1-2. (canceled)

3. A method of increasing penetration of a pharmacological agent across a blood-brain barrier in a subject, the method comprising
administering the pharmacological agent to the subject; and
providing at least one stimulus to a midbrain area of the subject, thereby increasing the activity of dopaminergic neurons in the midbrain area of the subject;
wherein increasing the activity of dopaminergic neurons in the midbrain area of the subject increases penetration of the pharmacological agent across the blood-brain barrier in the subject.

4. The method of claim 3, wherein the dopaminergic neurons are in a ventral tegmental area (VTA) of the midbrain or in a Substantia Nigra pars compacta of the midbrain.

5. (canceled)

6. The method of claim 3, wherein the at least one stimulus is a behavioral stimulus.

7. The method of claim 6, wherein the behavioral stimulus is rewarding.

8. The method of claim 6, wherein the behavioral stimulus leads to anticipation of a reward.

9. The method of claim 6, wherein the behavioral stimulus is positively surprising.

10. The method of claim 9, wherein a Reward Prediction Error is created.

11. The method of claim 6, wherein the behavioral stimulus creates invigoration in the subject.

12. The method of claim 6, further comprising providing a reward to the subject.

13. The method of claim 12, wherein the behavioral stimulus and/or the reward are provided to the subject based on the time of administration of the pharmacological agent so as to increase blood-brain barrier (BBB) permeability when the pharmacological agent reaches the BBB.

14. The method of claim 3, wherein the at least one stimulus is a deep brain stimulation, a trans-cranial magnetic stimulation (TMS), an optogenetic stimulation, or a chemogenetic stimulation.

15-17. (canceled)

18. The method of claim 3, wherein the at least one stimulus is a pharmacological agent that enhances the activity and/or efficacy of dopaminergic neuron activity.

19. The method of claim 3, wherein the at least one stimulus can emulate the action of dopaminergic neurons or of dopamine.

20. The method of claim 3, wherein the pharmacological agent is a therapeutic for treatment of a CNS disorder, pain, insomnia, stroke, or a combination thereof.

21. The method of claim 20, wherein the CNS disorder is depression, attention deficit hyperactivity disorder (ADHD), bipolar disorder, schizophrenia, Alzheimer's Disease, Parkinson's disease, memory deficit, cognition deficit, drug addiction, migraine, seizures, sleep disorders, cancer, vascular dementia, Lewy Body dementia (or dementia with Lewy Bodies), Frontotemporal dementia, Creutzfeldt-Jakob disease, Wernicke-Korsakoff syndrome, mixed dementia, normal pressure hydrocephalus, posterior cortical atrophy, Huntington's disease, down syndrome, and/or a combination thereof.

22. The method of claim 20, wherein the increased blood-brain permeability clears debris associated with the CNS disorder or stroke out of the brain.

23. (canceled)

24. The method of claim 22, wherein the BBB permeability is determined by measuring a change in influx of fluorescent molecules from vasculature into the brain.

25. The method of claim 22, wherein the BBB permeability is measured using human brain positron emission topography and/or magnetic resonance imaging methods.

26. A method of opening a blood-brain barrier, the method comprising:
providing at least one stimulus to a specific area of a brain; and
stimulating the specific area of the brain thereby opening the blood-brain barrier.

27. The method of claim 26, wherein the specific area of the brain is a ventral tegmental area (VTA) or a Substantia Nigra pars compacta.

28. (canceled)

29. The method of claim 26, wherein the at least one stimulus is a behavioral stimulus.

30. The method of claim 26, wherein stimulating the specific area of the brain increases ventral tegmental area dopamine neurons.

31. The method of claim 26, wherein stimulating the specific area of the brain increases Substantia Nigra pars compacta dopamine neurons.

32. A method of treating a disease, the method comprising:
providing at least one stimulus to a ventral tegmental area of a brain or to a Substantia Nigra pars compacta area of the brain; and
stimulating the ventral tegmental area of the brain or the Substantia Nigra pars compacta area of the brain to open a blood-brain barrier for the clearance of “junk” associated with the disease.

33-35. (canceled)
